(19) United States
(12) Patent Application Publication
(10) Pub. No.: US 2023/0037043 A1
(43) Pub. Date: Feb. 2, 2023

(54) Sound Counteracting System

(71) Applicants: Wendy Scheall-LaCroix, Wasilla, AK (US); Trish Jedlicka, Kenai, AK (US)

(72) Inventors: Wendy Scheall-LaCroix, Wasilla, AK (US); Trish Jedlicka, Kenai, AK (US)

(21) Appl. No.: 17/391,803

(22) Filed: Aug. 2, 2021

(51) Int. Cl. H04R 1/02; H04R 5/02; A61L 9/14

(52) U.S. Cl. CPC H04R 1/028; H04R 5/02; A61L 9/14; H04R 2420/07; A61L 2209/133

(57) ABSTRACT

The present invention is a system constructed and arranged to counteract bathroom sounds and scents including a housing, a speaker, microcomputer controller configured with input-output data mechanisms, a cartridge configured to store and expel a scent, a scent output, and a power source.

### BACKGROUND OF THE INVENTION

In the restroom, the acoustics often amplify the various sounds associated with bodily functions that occur therein.

There have been some attempts at countering, muffling, or otherwise hiding these sounds, but prior attempts have proven to have unsatisfactory effectiveness

The use of running water from facets, loud fans, and personal cell phone music apps have been the common ways to try and mask/hide these embarrassing yet uncontrollable but sometimes necessary noises, all while handling such things with the use of contaminated hands.

There is a need for a device to mask or hide these noises.

The present invention fulfills this need.

### SUMMARY OF THE INVENTION

In one embodiment the present invention is a system configured to block natural and normal sounds from escaping the privacy of one's restroom for others to hear.

In one embodiment, the invention is configured with a wireless Bluetooth speaker.

In one embodiment the device is a rectangular shaped water resistant box.

In use, in one embodiment, the device is mounted and centered above the door frame on the inside of the room. It is adhered to the wall by either hardware or double sided Velcro for easy placement and removal.

The device is configured as it projects acoustically balanced sounds to harmonize with and mask personal sounds within the restroom. In one embodiment, the device is configured with many different user selectable sound options (e.g., Amazon Rain Forest, Crashing Ocean Waves, White Noise, Midwestern Crackling Thunderstorm, April Showers, Cricket Summer Night, New Age Instrumentals, and more) with Bluetooth capabilities for a personal sound and music experience.

The device is activated upon entry by a motion detection sensor, and will automatically turn itself off upon exiting. This gives the occupant an instant curtain of privacy neutralizing personal sounds within the room.

The device is hard wired, battery powered, or is powered with both.

In one embodiment, the device is powered by lithium-ion technology.

In one embodiment, the device has LED status indicator lights to inform user of its current status, such as, on/off, battery low, and sound options.

In one embodiment, the device will use Bluetooth wireless technology so the user may opt to choose from their own variety of sounds and/or music via other devices.

In one embodiment, the invention is a system for counteracting bathroom sounds, said system comprising:
a housing;
a speaker associated with said housing;
a microcomputer controller configured with input-output data mechanisms;
a cartridge configured to store and expel a scent;
a scent output; and
a power source.

In one embodiment, the housing includes two speakers.

In one embodiment, a microcomputer controller is associated with storing non-transitory computer readable files.

In one embodiment, a microcomputer controller is associated with storing non-transitory computer readable files on flash memory.

In one embodiment, a microcomputer controller input-output receives and transmits information wirelessly.

In one embodiment, a cartridge is configured to contain scented solution.

In one embodiment, a scent output is configured to atomize scented solution from said cartridge.

In one embodiment, a cartridge is configured to contain scented spray.

In one embodiment, a power source is a hard wired source.

In one embodiment, a power source is a battery.

In one embodiment, a power source is a lithium ion battery.

In one embodiment, the invention is a system for counteracting bathroom sounds, said system consisting of:
a housing;
a speaker associated with said housing;
a microcomputer controller configured with input-output data mechanisms;
a cartridge configured to store and expel a scent;
a scent output; and
a power source.

### BRIEF DESCRIPTION OF THE SEVERAL VIEWS OF THE DRAWINGS

FIG. 1 is a perspective according to one embodiment of the present invention.

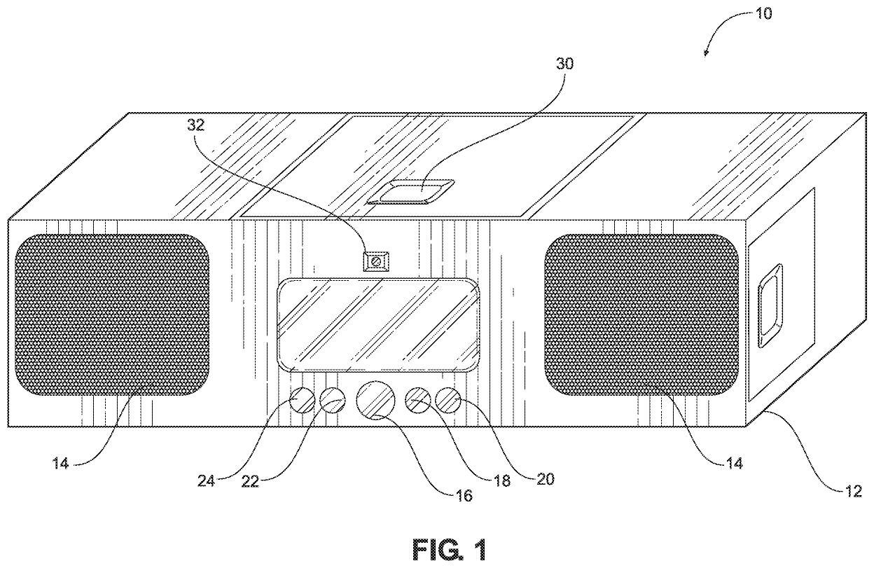

FIG. 2 demonstrates scent dispersion system according to one embodiment of the present invention.

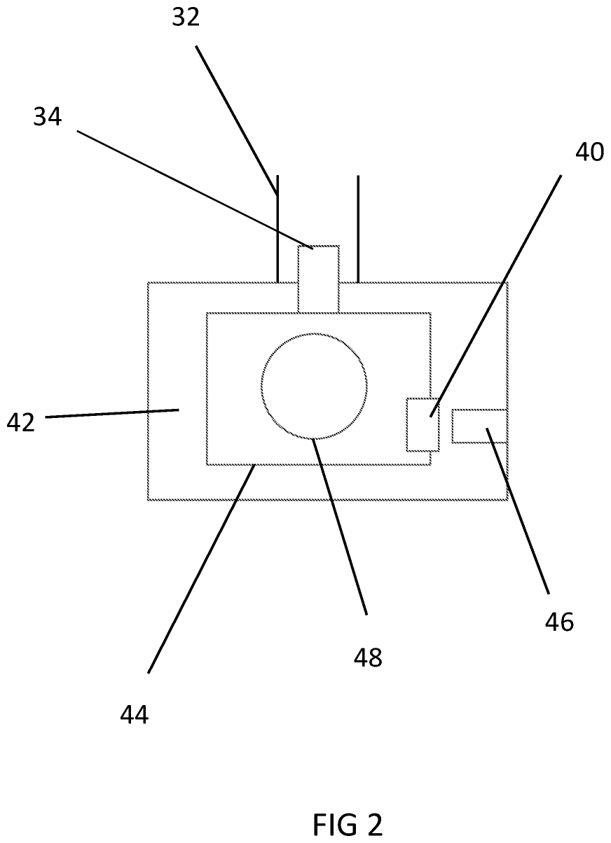

FIG. 3 is a schematic that demonstrates microcomputer control and processing according to one embodiment of the present invention.

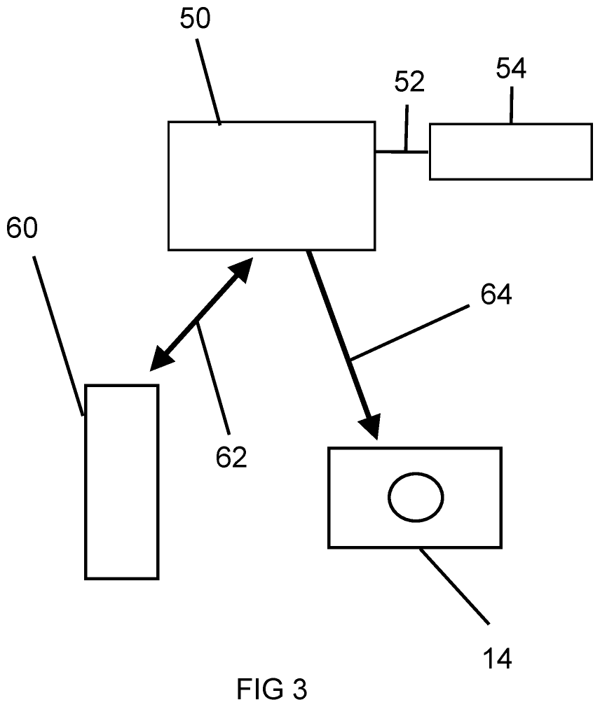

### DETAILED DESCRIPTION OF THE PREFERRED EMBODIMENTS

Although the figure provides an example of components according to one environment of the present invention, the inventive concept is not limited to this specific embodiment.

System 10 is contained in housing 12. Configured with housing 12 is audio speakers 14, motion sensor 16, first indicator light 18 second indicator light 20 third indicator light 22 and fourth indicator light 24. Any one or combination of the aforementioned indicator lights are configurable as a night light. The night lights are white and are also contemplated as configurable for customized colors.

Indicator lights are configured to provide information relating to power, Bluetooth connectivity, particular system mode, battery life, sent refill, and combinations thereof.

A cover 30 is placed over a storage compartment for a fragrance scent cartridge to be positioned therein. Housing 12 further includes scent spray nozzle 32 which provides for an exit when sent is to be expelled from system 10.

In use, housing 12 is positioned in a restroom as desired. In one embodiment, housing 12 is mounted to a wall inside a restroom. Mounting is accomplished through conventional means including but not limited to, permanent mounting, removable mounting, or positioning the device on a shelf as desired.

System 10 is constructed and arranged with appropriate electronics that will generate desired sounds audible through speakers 14. System 10 includes input output configurations whereby sounds are uploaded, stored and selected for use. As stated above, and without limitation sounds include but are not limited to Amazon Rain Forest, Crashing Ocean Waves, White Noise, Midwestern Crackling Thunderstorm, April Showers, Cricket Summer Night, and New Age Instrumentals. In a preferred embodiment, flash memory is included with system 10 whereby uploaded sound files are stored and played by the system as selected by user.

System 10 includes a controller device for selecting particular sounds, duration of sounds, metered and controlled amounts of scent, and combinations thereof.

As generally understood, when motion sensor 16 is actuated, speakers 14 are turned on and the selected sound emanates therefrom. The sounds continue for a preselected time and are automatically shut off once the time limit has been reached.

Underneath cover 30 is scent cartridge housing 42 in which scent cartridge 44 is positioned. System 10 has system actuator 46 configured to contact actuator receiver 40 that, upon contact, ultimately expels scent 48 through outlet 34 that exits system 10 through spray nozzle 32.

System 10 is configured with microprocessor 50 having a communications link 52 with computer storage 54 that is constructed and arranged to store files on non-transitory computer readable medium. A controller 60 is configured with input-output communications 62 to upload computer readable files to microprocessor 62. The files will typically be the sounds emanating from system 10. Microprocessor 50 has an output communications link 64 to speaker 14. In one embodiment, speaker 14 is incorporated into housing 12. In another embodiment, speaker 14 receives output from communications link 64 as a wireless signal and speaker 14 is remote and not incorporated into housing 12. In another embodiment, system 10 is configured with a combination one or more speakers incorporated within housing 12 and one or more speakers remote from housing 12.

A user will use controller 60 to upload and select files for output through speaker 14.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed:

1. A system for counteracting bathroom sounds, said system comprising:
a housing;
a speaker associated with said housing;
a microcomputer controller configured with input-output data mechanisms;
a cartridge configured to store and expel a scent;
a scent output; and
a power source.

2. The system of claim 1 wherein said housing includes two speakers.

3. The system of claim 1 wherein said microcomputer controller is associated with storing non-transitory computer readable files.

4. The system of claim 1 wherein said microcomputer controller is associated with storing non-transitory computer readable files on flash memory.

5. The system of claim 1 wherein said microcomputer controller input-output receives and transmits information wirelessly.

6. The system of claim 1 wherein said cartridge is configured to contain scented solution.

7. The system of claim 1 wherein said scent output is configured to atomize scented solution from said cartridge.

8. The system of claim 1 wherein said cartridge is configured to contain scented spray.

9. The system of claim 1 wherein said power source is a hard wired source.

10. The system of claim 1 wherein said power source is a battery.

11. The system of claim 1 wherein said power source is a lithium ion battery.
